(19) United States
(12) Patent Application Publication
(10) Pub. No.: US 2015/0083596 A1
(43) Pub. Date: Mar. 26, 2015

(54) Device and method for killing bacteria and viruses in blood

(71) Applicant: Dan Hester, Chattanooga, TN (US)

(72) Inventor: Dan Hester, Chattanooga, TN (US)

(21) Appl. No.: 14/496,861

(22) Filed: Sep. 25, 2014

Related U.S. Application Data
(60) Provisional application No. 61/882,619, filed on Sep. 25, 2013.

(51) Int. Cl. A61M 1/36; A61M 39/22

(52) U.S. Cl. CPC A61M 1/3681; A61M 39/22; A61M 1/3672; A61B 2018/00398
USPC 204/550; 204/600

(57) ABSTRACT

A device, system and method for killing viruses and bacteria in blood. An iontophoretic cartridge destroys blood borne viruses and bacteria using ionized silver nanoparticles. Blood from the arm of the patient is routed to a holding bladder. From the bladder, the blood is pumped to the iontophoretic cartridge. In the cartridge, the blood is split into four tubes containing silver nanowires that treat the blood and destroy the viruses as they flow through the cartridge. The blood is then rerouted to the patient's arm.

### CROSS REFERENCE TO RELATED APPLICATIONS

This application claims the benefit of U.S. Provisional Patent App. No. 61/882,619 filed on Sep. 25, 2013, the entirety of which is hereby incorporated by reference.

### STATEMENT REGARDING FEDERALLY SPONSORED RESEARCH OR DEVELOPMENT

### BACKGROUND OF THE INVENTION

This invention was not federally sponsored.

### Field of the Invention

This invention relates to the general field of medical devices and methods, and more specifically toward a device, system and method for killing viruses and bacteria in blood. An iontophoretic cartridge destroys blood borne viruses and bacteria using ionized silver nanoparticles. Blood from the arm of the patient is routed to a holding bladder. From the bladder, the blood is pumped to the iontophoretic cartridge. In the cartridge, the blood is split into a plurality of tubes containing silver nanowires that treat the blood and destroy viruses and/or bacteria as they flow through the cartridge. The blood is then rerouted to the patient's arm.

Viral infections of the blood can have a devastating impact on a patient once the virus has infected its host. In many cases it ends with the death of the host. The more deadly forms of the blood viruses are HIV/AIDS and Hepatitis C. HIV can mutate quickly and therefore can be very difficult to create an effective vaccine or drug cocktail that works against it. With Hepatitis C, there are many different strains of the virus and therefore drug therapies that are available can be non-effective and/or have debilitating side effects. The patients that the drugs do not work on are classified as non-responders and there chances for survival are very bleak. One particular strain of Hepatitis C has been linked to a new form of liver cancer that has not been seen before. There has also been a rise in Hepatitis C worldwide due to the popularity of tattooing and the lack of proper hygiene associated with it.

Silver iontophoresis is a physical process wherein silver ions are driven by an electrical field and flow diffusively through a medium. The prior art has used silver iontophoresis by inserting a catheter into the subclavian vein or the superior vena cava and then placing a silver probe through it and directly into the blood stream. A small electrical current is then applied to the wire in the prescribed amount to release the proper amount of silver nanoparticles, which have a slightly positive charge, to bond to viruses, which has a slightly negative charge. This process destroys the virus by disrupting the functions of the membrane of the virus and thus its ability to survive. This procedure has various challenges associated with it due to its close proximity to the heart, its duration of time needed to be successful and its chances of creating a secondary infection at the entry site.

Thus there has existed a long-felt need for a device and method that efficiently and safely kills viruses in blood.

### SUMMARY OF THE INVENTION

The current invention provides just such a solution by having a device, system and method for killing viruses and/or bacteria in blood. An iontophoretic cartridge destroys blood borne viruses and bacteria using ionized silver nanoparticles. Blood from the arm of the patient is routed to a holding bladder. From the bladder, the blood is pumped to the iontophoretic cartridge. In the cartridge, the blood is split into a plurality of tubes, for example four tubes, containing silver nanowires that treat the blood and destroy the viruses, bacteria, and/or other microbes as they flow trough the cartridge. The blood is then rerouted to the patients arm.

What is being developed is a machine that is fitted with a disposable cartridge that will collect blood from the body and treat the blood externally rather than internally. The machine will collect blood from the arm of the patient and route the blood to a holding bladder. From the bladder the blood will be pumped into the iontophoretic cartridge. Once inside the cartridge the blood will be split into four specially designed tubes containing silver nanowires that will treat the blood and destroy the viruses and/or bacteria as they flow trough the cartridge. When an electrical current, charge, or field is applied to the aleated silver rods, silver ions are discharged into the blood stream through electrolysis. Appropriate concentrations and durations of silver ions in blood can kill viruses, bacteria, and/or other microbes without affecting eukaryotic cells, such as erythrocytes (red blood cells) and leukocytes (white blood cells).

The blood will then be rerouted to the patient's arm. The machine will: collect the blood, heat the blood, pump the blood trough the machine and back to the patient, monitor and maintain proper blood pressure and heart rate, be able to administer anticoagulant if needed, monitor and remove air, and monitor all vital signs in real time and send network alerts if needed. This process will greatly reduce the exposure to life threatening complications as well as a substantial reduction in duration of time needed for the treatment. The procedure could be performed at a doctor's office, at home, at the hospital, or as a vacation extended treatment.

The electrical system for the iontophoretic cartridge is a multi-power source configuration with various potential sources of power, including both alternating and direct current. For example, power sources may include power from batteries, the power grid, solar cells, and automotive batteries and alternators. An AC-DC power converter is available to convert alternating current to direct current, as well as modify the voltage level to operate appropriately. Allowing for various different power sources enables the device and method to respond to varying environments it will be operating in.

It is an object of the invention to provide a device for killing viruses, bacteria, and/or other microbes in blood.

It is another object of the invention to provide a method for killing viruses, bacteria, and/or other microbes in blood.

It is a further object of this invention to provide a device and method for treating blood externally from the body.

As used herein, the term “patient” refers not only to a human, but also to mammals and even animals in general; the terms “rod” or “wire” refer to a thin, straight, and rigid or flexible bar; the term “aleated” means coated or insulated except for a small portion. Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term.

Terms and phrases used in this document, and variations thereof, unless otherwise expressly stated, should be construed as open ended as opposed to limiting. As examples of the foregoing: the term “including” should be read as meaning “including without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein may be described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

In a particular embodiment, the current invention is a device for treating a viral infection within a blood stream comprising an iontophoretic cartridge, where the iontophoretic cartridge comprises a plurality of silver rods; a transformer, where the transformer comprises a plurality of resistors and a transducer, where the transformer is in electrical connection with the iontophoretic cartridge; a blood pump; an arterial pressure monitor; a heat exchanger; a venous pressure monitor; and an air trap/detector; where the iontophoretic cartridge, blood pump, arterial pressure monitor, heat exchanger, venous pressure monitor and air trap/detector are in fluid connection.

In another embodiment, the current invention is a method for killing a virus within a patient comprising the steps of removing blood from the patient; passing the blood through an iontophoretic cartridge, where the iontophoretic cartridge comprises a plurality of silver rods; providing electrical current to the iontophoretic cartridge; and returning the blood to the patient.

In yet another embodiment, the current invention is an iontophoretic cartridge comprising a main chamber, where the main chamber comprises a plurality of tubes, where each tube comprises a silver rod, where each tube has a diameter of one-half of an inch; a blood input port; a blood output port; a blood collection valve; a plurality of resistors; and an anticoagulant valve.

A further embodiment of the current invention is a system comprising an iontophoretic cartridge, where the iontophoretic cartridge comprises a plurality of aleated silver rods and a blood collection valve; an electrical power source connected to the iontophoretic cartridge; a pump, where the pump is in fluid connection with the iontophoretic cartridge; and a pressure sensor, where the pressure sensor detects the pressure of a fluid before it enters the iontophoretic cartridge. The system further comprises an arterial pressure sensor, where the arterial pressure sensor detects the pressure of a fluid before it enters the pump. The system further comprises a venous pressure sensor, where the venous pressure sensor detects the pressure of a fluid after it leaves the iontophoretic cartridge. The fluid is blood. The system further comprises an air sensor, where the air sensor detects the presence of air within the blood after it exits the iontophoretic cartridge. The system further comprises an oxygen saturation sensor, where the oxygen saturation sensor detects the saturation of oxygen within the blood after it exits the iontophoretic cartridge. The system further comprises a heat exchange, where the heat exchanger is fluidly connected to the iontophoretic cartridge.

An additional embodiment of the current disclosure provides for a device comprising a blood input adapter; a chamber, where the chamber comprises a plurality of aleated silver rods; a blood output adapter; a blood collection valve; an anti-coagulant valve, and an electrical connector; where the blood input adapter, blood output adapter, blood collection valve, and anti-coagulant valve are fluidly connected to the chamber, and where the electrical connector is electrically connected to the aleated silver rods. The electrical connector comprises a positive terminal and a negative terminal, or the electrical connector comprises two metallic collars. The device further comprises an anode, where the anode is electrically connected to the electrical connector. The device further comprises a cathode, where the cathode is electrically connected to the electrical connector. The device further comprises four resistors, where the four resistors are electrically connected to the electrical connector and the plurality of aleated silver rods. Each of the four resistors has a resistance of 5.8 kilo-ohms or more, and where each of the four resistors has a resistance of 3.3 mega-ohms or less.

Another embodiment of the current disclosure provides for a method of treating blood for infections comprising the steps of pumping blood through an iontophoretic cartridge, where the iontophoretic cartridge comprises a plurality of aleated silver rods; and providing an electrical current to the iontophoretic cartridge while the blood is pumped through the iontophoretic cartridge. The method further comprises the step of detecting a pressure value of the blood before it enters the iontophoretic cartridge. The method further comprises the step of detecting a pressure value of the blood before it is pumped through the iontophoretic cartridge. The method further comprises the step of detecting a pressure value of the blood after it exits the iontophoretic cartridge. The method further comprises the step of detecting the presence of air within the blood after the blood exits the iontophoretic cartridge. The iontophoretic cartridge further comprises an anti-coagulant valve; wherein the method further comprises the step of administering anticoagulants to the blood as it is pumped through the iontophoretic cartridge.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims.

### BRIEF DESCRIPTION OF THE FIGURES

The accompanying drawings, which are incorporated in and form a part of this specification, illustrate embodiments of the invention and together with the description, serve to explain the principles of this invention.

FIG. 1 is a schematic view of a device and method for killing bacteria, viruses, and/or other microbial organisms in blood of a patient according to selected embodiments of the current disclosure.

FIG. 2 is a schematic view of an iontophoretic cartridge according to selected embodiments of the current disclosure.

FIG. 3 is a diagram of electrical components according to selected embodiments of the current disclosure.

FIG. 4 is a diagram of a device for killing bacteria, viruses, and/or other microbial organisms in blood according to selected embodiments of the current disclosure.

### DETAILED DESCRIPTION OF THE INVENTION

Many aspects of the invention can be better understood with the references made to the drawings below. The components in the drawings are not necessarily drawn to scale. Instead, emphasis is placed upon clearly illustrating the components of the present invention. Moreover, like reference numerals designate corresponding parts through the several views in the drawings.

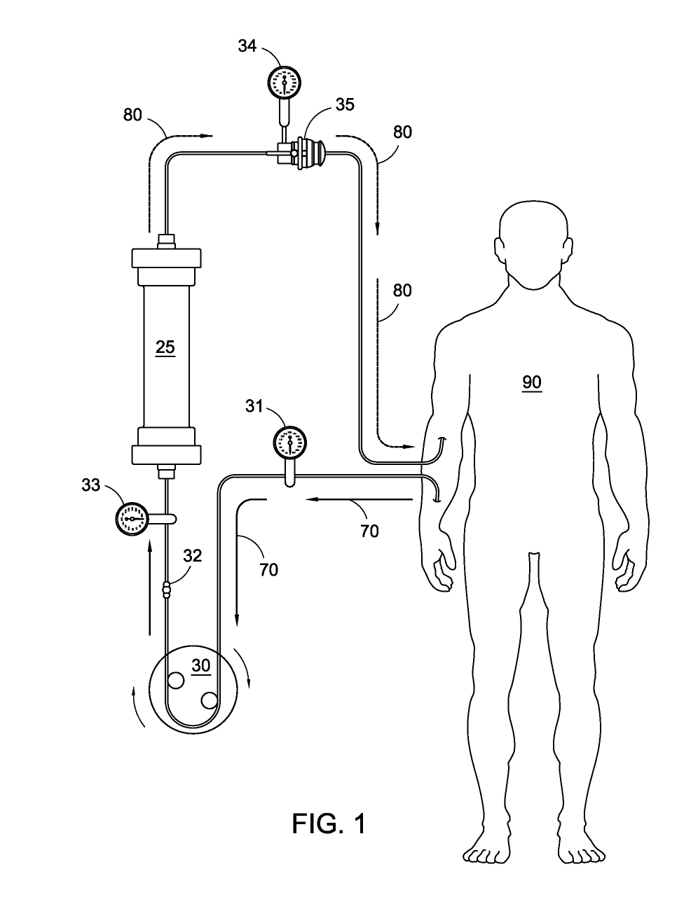

FIG. 1 is a schematic view of a device and method for killing bacteria and viruses in blood of a patient according to selected embodiments of the current disclosure. Untreated blood 70 is removed from patient 90. While it is shown that the untreated blood 70 is removed from the arm of the patient 90, it is nonetheless possible and in some cases may be preferably to remove untreated blood 70 from another location on the patient 90. A blood pump 30 is used to withdraw the untreated blood 70 from the patient 90 and move it through the rest of the system. An arterial pressure monitor 31 displays the pressure of the untreated blood 70 removed for treatment. A heat exchange 32 is used to transfer heat to the blood to increase its temperature as it travels through the system. A pressure monitor or blood pressure sensor 33 detects the pressure of the untreated blood 70 before it enters the iontophoretic cartridge 18.

The iontophoretic cartridge 25 includes silver rods 18. Electric current running through the silver rods causes the silver rods 18 to release ionized silver nanoparticles into the blood thereby killing the viruses. In a particular embodiment, a five-microampere power source is used to supply electrical current to the silver rods 18 in the iontophoretic cartridge 25.

Treated blood 80 leaves the iontophoretic cartridge 25 and passes through an air trap/air detector 35 before returning to the patient 90. A venous pressure monitor 34 detects the pressure of the treated blood 80 after it leaves the iontophoretic cartridge 25. As with removing untreated blood 70 from the patient 90, FIG. 1 shows treated blood 80 returning to the arm of the patient 90, though it is possible and in some cases may be preferable to return treated blood 80 to another location on the patient 90. In a particular embodiment, blood flows through tubing, such as surgical tubing, between and/or through the various elements in the system and method described herein.

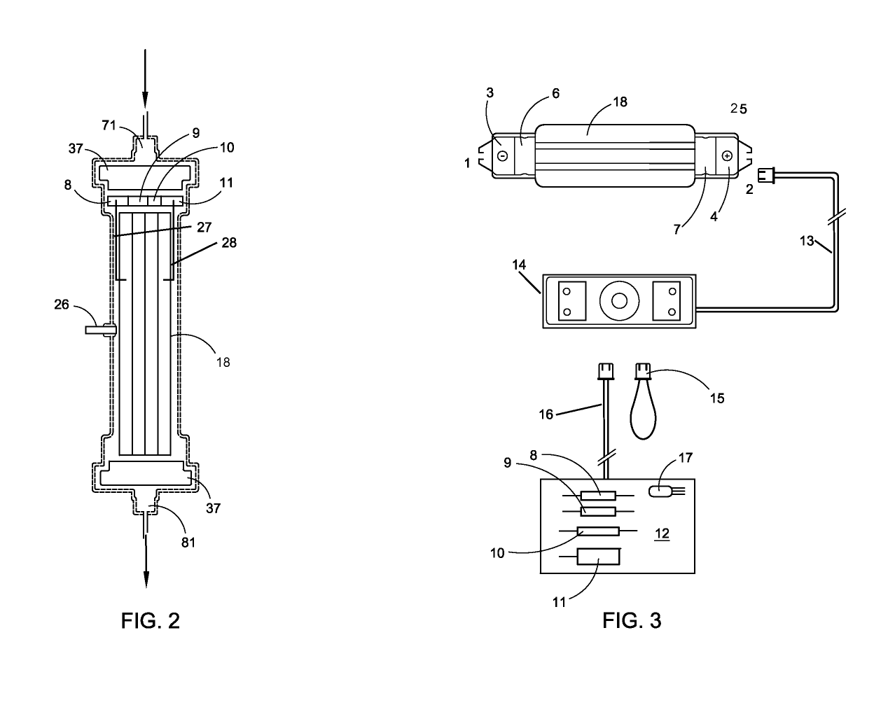

FIG. 2 is a schematic view of an iontophoretic cartridge according to selected embodiments of the current disclosure. Blood is supplied to the iontophoretic cartridge 25 through blood input adapter 71. The blood then travels through blood collection valve 37, which helps maintain blood pressure within the system and prevents the backflow of blood. The blood then travels through a main chamber 19 that houses four silver rods 18 housed within ½-inch tubes. An electrical power source is connected to the iontophoretic cartridge to cause an electrical current to travel through the silver rods 18. Resistors 8, 9, 10, and 11, described in more detail below, regulate the electrical current that travels through anode 27 and cathode 28 and the silver rods 18. An anticoagulant valve 26 is provided to administer anticoagulants into the blood as it travels through the iontophoretic cartridge 25.

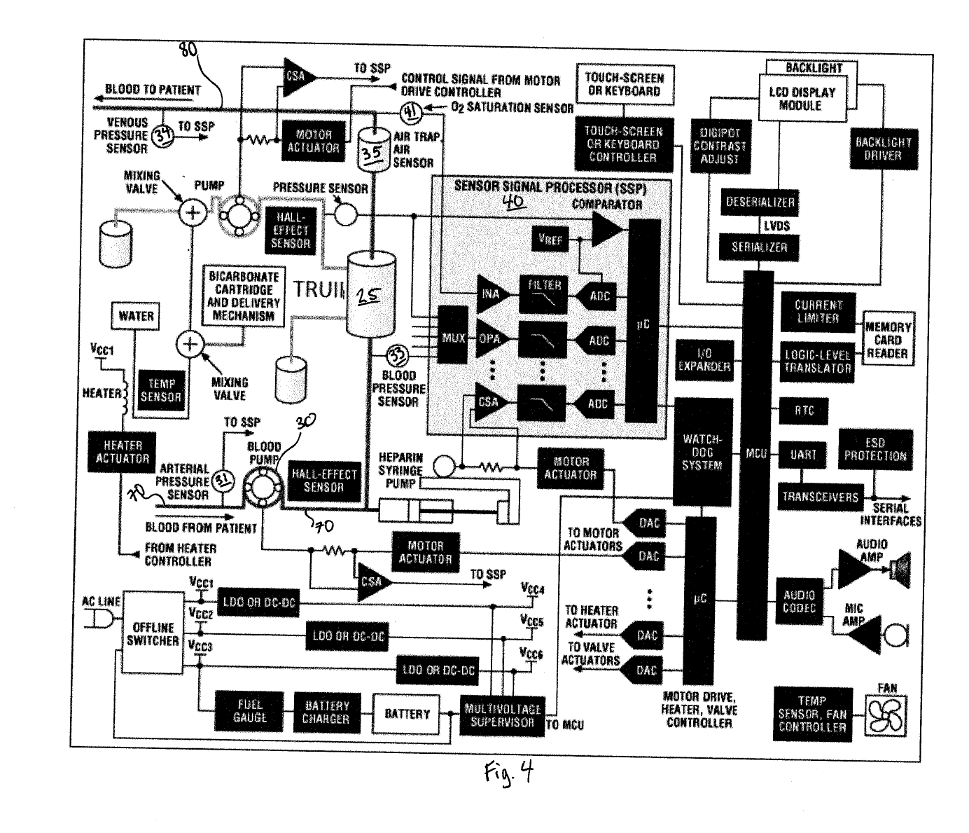

FIG. 3 is a diagram of electrical components according to selected embodiments of the current disclosure. Entry point 1 and exit point 2 show where the blood flows through the cartridge. Negative terminal lug 3 and positive terminal lug 4 are used in the event the connectors are not feasible; cathode and anode wires can be attached to these lugs in order to supply power. The wire coming from the negative lug is grounded to a housing of any machine in which the cartridge is placed. Recessed female plug joins with a male connector to allow the cartridge to draw power from a vehicle such as through cigarette lighter port. Metallic collars 6 and 7 receive power from positive and negative clamps when the cartridge is inserted in to a specific type of machine. This assembly will negate the need for wires or connectors. Two-port power connector 14 gives the cartridge versatility to connect to other types of power sources. End cap 15 gives the cartridge seamless integration into an array of different machines. As will be appreciated by those skilled in the art, the different forms of electrical connections described above can be used individually or in combination with others in the same iontophoretic cartridge.

A transformer 12 may be used to convert an external power source to a power source suitable for the iontophoretic cartridge. The transformer may include integrated resistors 8, 9, 10 and 11, which play an integral part in the makeup of particular embodiments of the iontophoretic cartridge or iontophoretic cartridge system as they regulate the electrical current and keep it between five (5) microamperes and ten (10) microamperes. Resistor 8 is a 5.8 kilo-ohm (kΩ) ¼-watt resistor. Resistor 9 is a 3.3 mega-ohm (mΩ) ¼-watt resistor. Resistor 10 is a 1 mega-ohm (mΩ) ¼-watt resistor. Resistor 11 is a 150 kilo-ohm (kΩ) ¼-watt resistor. For applications where a higher current is required, transistor 17 is incorporated into the transformer or iontophoretic cartridge. Transistor 17 is a Darlington Transistor MPS A64-PNP that is used for applications that require very high values of current, such as higher than 800 mA. A twenty-two (22) AWG double lead 16 is used to electrically connect the transformer to the two-port power connector 14. In a particular embodiment, the double lead 16 is seventeen (17) inches long.

A 78-inch 24 American wire gauge (AWG) lead may be used to accommodate any reasonable scenario that requires a long stretch to a power source. Excess wire can be folded and secured by various means well known in the art, such as wire ties, zip ties, and hook and loop fastening straps.

The power provided to the iontophoretic cartridge flows through four aleated rods 18. Current causes the rods to release ionized silver nanoparticles in to the blood thereby killing the viruses. In a particular embodiment, each aleated rod is coated with an insulating material except for a ½-¾ inch portion at one end of the rod.

FIG. 4 is a diagram of a device for killing bacteria and viruses in blood according to selected embodiments of the current disclosure. A blood pump 30 pumps untreated blood 70 from a patient to an iontophoretic cartridge 25. An arterial pressure sensor 31 senses the pressure of the untreated blood 70 before it passes through the blood pump 30. The arterial pressure sensor 31 is connected to a sensor signal process 40, which processes signals from the various sensors of the device. A blood pressure monitor or sensor 33, also connected to the sensor signal processor 40, senses the pressure of the untreated blood 70 before it enters the iontophoretic cartridge 25. Electrical current provided to the iontophoretic cartridge causes the aleated silver rods contained therein to release ionized silver nanoparticles into the blood thereby killing viruses and/or bacteria. The treated blood 80 leaves the iontophoretic cartridge 25 and passes through/by an air trap/air detector or sensor 35. An oxygen saturation sensor 41, which is connected to the sensor signal processor 40, measures the oxygen saturation of the treated blood 80. Additionally, a venous pressure sensor 34, which is connected to the sensor signal processor 40, senses the pressure of the untreated blood 80 before it is returned to the patient. The sensor signal processor 40 accepts signals obtained from the various sensors to generate values related to that sensor, including pressure values and oxygen saturation values.

It should be understood that while the preferred embodiments of the invention are described in some detail herein, the present disclosure is made by way of example only and that variations and changes thereto are possible without departing from the subject matter coming within the scope of the invention.

That which is claimed:

1. A system comprising
an iontophoretic cartridge, where the iontophoretic cartridge comprises a plurality of aleated silver rods and a blood collection valve;
an electrical power source connected to the iontophoretic cartridge;
a pump, where the pump is in fluid connection with the iontophoretic cartridge;
a pressure sensor, where the pressure sensor detects the pressure of a fluid before it enters the iontophoretic cartridge.

2. The system of claim 1, further comprising an arterial pressure sensor, where the arterial pressure sensor detects the pressure of a fluid before it enters the pump.

3. The system of claim 1, further comprising a venous pressure sensor, where the venous pressure sensor detects the pressure of a fluid after it leaves the iontophoretic cartridge.

4. The system of claim 1, wherein the fluid is blood.

5. The system of claim 4, further comprising an air sensor, where the air sensor detects the presence of air within the blood after it exits the iontophoretic cartridge.

6. The system of claim 4, further comprising an oxygen saturation sensor, where the oxygen saturation sensor detects the saturation of oxygen within the blood after it exits the iontophoretic cartridge.

7. The system of claim 1, further comprising a heat exchange, where the heat exchanger is fluidly connected to the iontophoretic cartridge.

8. A device comprising
a blood input adapter;
a chamber, where the chamber comprises a plurality of aleated silver rods;
a blood output adapter;
a blood collection valve;
an anti-coagulant valve, and
an electrical connector;
where the blood input adapter, blood output adapter, blood collection valve, and anti-coagulant valve are fluidly connected to the chamber, and where the electrical connector is electrically connected to the aleated silver rods.

9. The device of claim 8, wherein the electrical connector comprises a positive terminal and a negative terminal.

10. The device of claim 8, wherein the electrical connector comprises two metallic collars.

11. The device of claim 8, further comprising an anode, where the anode is electrically connected to the electrical connector.

12. The device of claim 8, further comprising a cathode, where the cathode is electrically connected to the electrical connector.

13. The device of claim 8, further comprising four resistors, where the four resistors are electrically connected to the electrical connector and the plurality of aleated silver rods.

14. The device of claim 13, where each of the four resistors has a resistance of 5.8 kilo-ohms or more, and where each of the four resistors has a resistance of 3.3 mega-ohms or less.

15. A method of treating blood for infections comprising the steps of
pumping blood through an iontophoretic cartridge, where the iontophoretic cartridge comprises a plurality of aleated silver rods; and
providing an electrical current to the iontophoretic cartridge while the blood is pumped through the iontophoretic cartridge.

16. The method of claim 15, further comprising the step of detecting a pressure value of the blood before it enters the iontophoretic cartridge.

17. The method of claim 15, further comprising the step of detecting a pressure value of the blood before it is pumped through the iontophoretic cartridge.

18. The method of claim 15, further comprising the step of detecting a pressure value of the blood after it exits the iontophoretic cartridge.

19. The method of claim 15, further comprising the step of detecting the presence of air within the blood after the blood exits the iontophoretic cartridge.

20. The method of claim 15, wherein the iontophoretic cartridge further comprises an anticoagulant valve; wherein the method further comprises the step of administering anticoagulants to the blood as it is pumped through the iontophoretic cartridge.
